(19) United States
(12) Patent Application Publication
(10) Pub. No.: US 2016/0317085 A1
(43) Pub. Date: Nov. 3, 2016

(54) NONINVASIVE SYSTEM AND METHODS FOR UTILIZING IMPEDANCE FOR THE DETECTION OF CEREBROSPINAL FLUID VOLUME

(71) Applicant: SARANAS, INC., Houston, TX (US)

(72) Inventors: Mehdi RAZAVI, Houston, TX (US); Anand Veerasakthy GANAPATHY, Houston, TX (US); Christopher Alexander AREVALOS, Houston, TX (US); Andrew Peyton FISHER, Austin, TX (US); Shelby Elaine HICKS, Tomball, TX (US); Lars Emerson LEIGHTON KUSLICH, Liberty, TX (US); Okenwa Chukwuemeka OKOSE, Friendswood, TX (US); Marianne BEYNON, Houston, TX (US); Momona YAMAGAMI, Houston, TX (US); Akshat JARIWALA, Houston, TX (US)

(21) Appl. No.: 15/142,506

(22) Filed: Apr. 29, 2016

Related U.S. Application Data
(60) Provisional application No. 62/155,302, filed on Apr. 30, 2015.

(51) Int. Cl. A61B 5/00; A61B 5/053

(52) U.S. Cl. CPC A61B 5/4881; A61B 5/6803; A61B 5/0537; A61B 5/4064; A61B 5/742; A61B 5/0004; A61B 2560/0223; A61B 2560/0214

(57) ABSTRACT

A cerebrospinal fluid (CSF) detection device includes a headband including at least four electrodes integrated into the headband. The electrodes are configured to make electrical contact with a person's head when the headband is placed on the person's head. The CSF detection device also includes an impedance circuit integrated into the headband and coupled to the at least four electrodes. The impedance circuit includes memory and an impedance analyzer coupled to the at least four electrodes and is configured to measure an impedance of the person's head. A first pair of the at least four electrodes is used by the impedance circuit to inject an electrical signal into the person's head and a second pair of the electrodes are used by the impedance analyzer to sense a signal. The first pair of electrodes is interleaved within the headband with respect to the second pair of electrodes.

### CROSS-REFERENCE TO RELATED APPLICATIONS

This application claims priority to U.S. Provisional Patent Application Ser. No. 62/155,302 entitled “Noninvasive System and Methods for Utilizing Impedance for the Detection of Cerebrospinal Fluid Volume,” filed Apr. 30, 2015 and incorporated herein by reference.

### BACKGROUND

Hydrocephalus is a disorder that is characterized by the accumulation of cerebrospinal fluid (CSF) in the brain. One of the management strategies to address this condition is through the use of ventriculoperitoneal (VP) shunts, which drain excess CSF from the ventricles of the brain into the peritoneal space in the abdomen. The procedure for implanting a VP shunt includes insertion of two sets of catheters. One is threaded into the brain through a small hole drilled into the skull. The other is placed subcutaneously from the ear to the abdomen, where the CSF is drained into the abdominal cavity. The two catheters are connected with a valve. The valve allows CSF to pass through the catheters when pressure in the brain becomes too high.

Over time VP shunts tend to fail from a myriad of issues most commonly via infection, mechanical failure or by obstruction due to tissue growth into the catheter. When this occurs CSF re-accumulates in the brain. The current solution to VP shunt failures is a subsequent procedure to install a new VP shunt.

The major problem with VP shunt failures is that they are difficult to diagnose. Failure is most commonly detected after symptoms of hydrocephalus become apparent. The symptoms of hydrocephalus include head swelling (mostly seen in infants), vomiting, nausea, sleepiness, irritability, headache, seizures, memory loss, and many more. However, these symptoms are nonspecific and can be difficult to distinguish from an isolated event of any of these symptoms (e.g., a viral illness). Current methods for confirmatory diagnosis of VP shunt failure include sophisticated and costly hospital-based imaging techniques such as magnetic resonance imaging (MRI) or computed tomography (CT) scans.

### BRIEF SUMMARY

The disclosed embodiments use a set of electrodes and corresponding circuitry to detect VP shunt failure in the cranium. By sending an electric current through the brain tissue and ventricles, and measuring the voltage drop across them, a bioimpedance can be calculated. Accumulation of CSF in the cranium due to VP shunt failure is correlated with bioimpedance changes. The disclosed apparatus uses this phenomenon to allow at monitoring of changes in VP shunt failure status due to intracranial fluid volume changes. A head strap containing multiple sets of electrodes will be placed on the patient's head. In some embodiments, one or more of the electrodes produce a sinusoidal electric current while other electrodes are used to measure the resulting voltage difference across the head. In such embodiments, by using different pairs of electrodes for transmitting current and measuring voltage instead of just two, the effect of the voltage drop caused by the electrodes is lessened due to the high resistance of the measuring side of the circuit. Therefore, only the voltage drop caused by the bioimpedance is measured, allowing for a more accurate detecting of VP shunt failure. Each device will be calibrated to the individual patient and a threshold level will be set to warn the user that CSF is accumulating within the brain indicating possible VP shunt failure might have occurred. The device can display data regarding a possible CSF accumulation on an integrated monitor, or wirelessly transfer such data to an external monitor where a message can be displayed.

### BRIEF DESCRIPTION OF THE DRAWINGS

For a detailed description of the disclosed embodiments, reference will now be made to the accompanying drawings in which:

FIG. 1 shows a side view of a patient wearing a CSF detection device in accordance with various embodiments;

FIG. 2 shows a front view of the CSF detection device in accordance with various embodiments;

FIG. 3 shows a side perspective view of the CSF detection device in accordance with various embodiments;

FIG. 4 shows a block diagram of the CSF detection device in accordance with various embodiments; and

FIG. 5 illustrates the CSF detection device in wireless communication with an external device in accordance with various embodiments.

### DETAILED DESCRIPTION OF EXEMPLARY EMBODIMENTS

As explained above, many children and adults suffering from hydrocephalus require placement of VP shunts to remove this excess fluid from the ventricles in their brains. Without the VP shunt, CSF would accumulate creating excess pressure on the surrounding brain tissue. Unfortunately, these shunts can fail as a result of tissue growth covering the openings of the shunt itself or for other reasons. Current medical practitioners specializing in neurology/neurosurgery lack the proper tools needed to determine VP shunt failures without the use of expensive imaging procedures, such as CT or MRI. This leads to many patients making multiple unnecessary hospital trips for symptoms that most commonly are benign in nature. The device described herein can be used to help patients determine more objectively whether a trip to the hospital is justified or if imaging is needed. In accordance with the disclosed embodiments, the device measures bioimpedance to detect an accumulation of fluid in the ventricles. The described approach uses a non-invasive extracortical sensor that, when placed on the patient's head, can be used to determine the presence or absence of shunt failure by monitoring changes in the bioimpedance of the region of interest in the cranium. The device may then calculate the pressure in the ventricles of the brain and use the calculated pressure value to generate an alert for any possible medical emergencies involving CSF accumulation.

Various embodiments are described herein for measuring intracranial impedance to detect an excess accumulation of CSF. Impedance may be computed by injecting a known current (DC or AC) and measuring the resulting voltage, or imposing a known voltage across the electrodes and measuring the resulting current. The ratio of voltage to current can be used to determine impedance. A baseline impedance reading is taken while it is known that CSF levels in the ventricles are normal for the specific patient and that the VP shunt is working correctly. Periodic impedance measurements can then be taken (e.g., once per day, once per week, etc.). If a patient's VP shunt fails, CSF will begin to accumulate in the patient's ventricles. As a result, the impedance of the cranium and its contents will decrease. The device can determine whether the impedance measurement is trending downward and/or has dropped below a threshold impedance level indicative of a VP shunt failure.

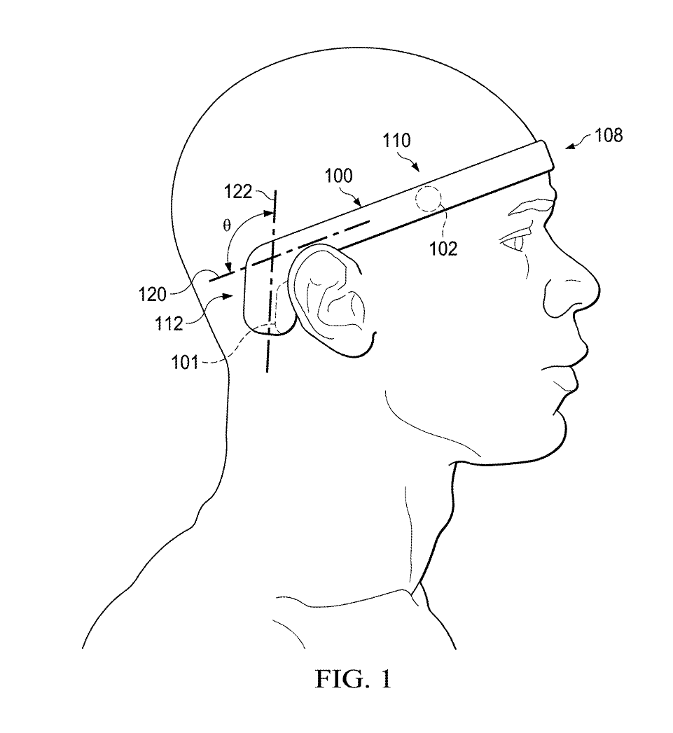

FIG. 1 illustrates a patient wearing a CSF detection device 100 in accordance with the disclosed embodiments. The CSF detection device generally comprises a headband configured to fit across the patient's forehead and temples and wraps around the patient's ears. The headband may be formed as one continuous piece (or separate pieces coupled together) but generally includes a forehead segment 108, a pair of temple portions 110, and a pair of temple tips 112. In the disclosed embodiment, the CSF detection device 100 includes at least three electrodes integrated into the headband at various locations along an interior surface of the headband so as to make direct electrical contact with the patient's head, but could include four or more electrodes in other embodiments. In the disclosed embodiment, the headband includes four electrodes. Each temple tip 112 includes an electrode such as electrode 101 shown in FIG. 1. Further, each of the temple portions 110 includes an electrode as well, such as electrode 102 as shown.

In some embodiments, the headband is not one continuous loop and, instead, is configured to fit about a partial circumference of the person's head. In other embodiments, the headband may completely wrap around the person's head.

The CSF detection device 100 includes a battery (disposable or rechargeable) and a circuit that are integrated into the headband. The circuit includes a signal generator that causes a current to be generated and injected between a pair of the electrodes and through the patient's cranium. Another pair of electrodes on the headband is also connected to the signal generator, and the circuit measures the voltage across those electrodes that results from the current. The ratio of voltage to current can be computed and represents impedance. Changes in impedance can be correlated to excessive accumulation of CSF in the person's ventricles.

Referring still to FIG. 1, the temple tips 112 may comprise longitudinal portions that have a longitudinal axis 122. The temple portions 110 also have a longitudinal axis 120. The temple tips 112 are provided at an angle to the temple portions 110. The angle between the longitudinal axes 120 and 122 is designated in FIG. 1 as ⊖. The angle ⊖ may be between about 80 degrees and about 120 degrees in some embodiments. Because the temple tips 112 angle downward from temple portion axis 120, the electrodes (e.g., electrode 101) integrated into the temple tips will fit flush against the patient's skin just behind the ear and the patient's hair will not impedance sufficient electrical contact between electrode and skin. Similarly, the electrodes positioned along the temple portions 110 (e.g., electrode 102) also fit flush against the patient's skin along the patient's temples, generally below the hairline.

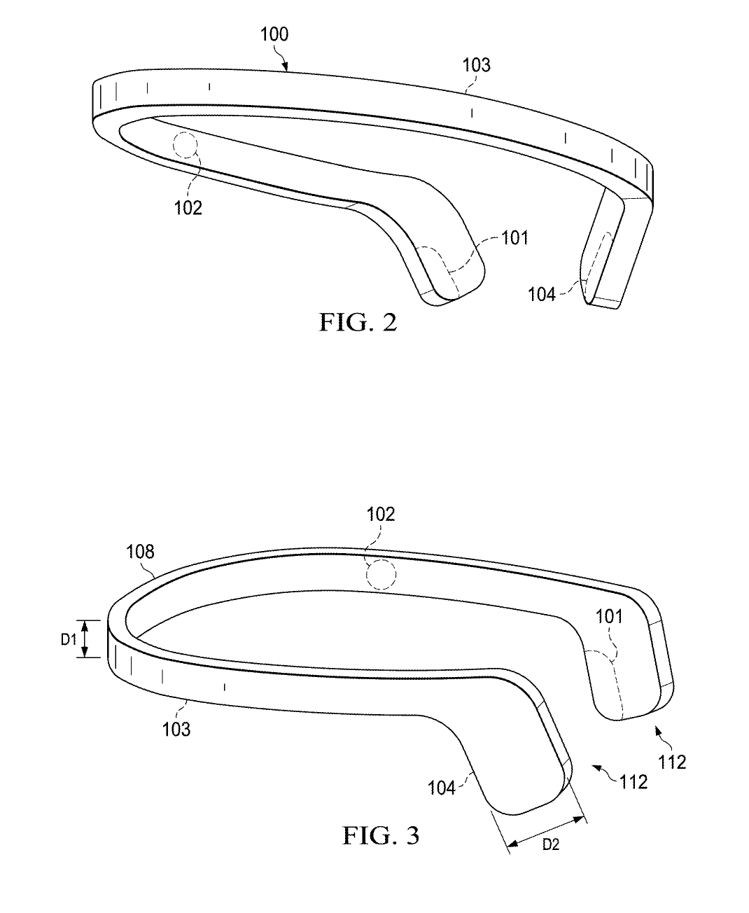

FIGS. 2 and 3 illustrate different views of the CSF detection device 100. The various electrodes 101-104 can be seen integrated into the headband. In the example shown, the width D1 of the forehead segment 108 is generally larger than the width D2 of the temple tips 112. The relatively larger width D2 of the temple tips 112 facilitates enhanced comfort to the patient and helps prevent the device from slipping.

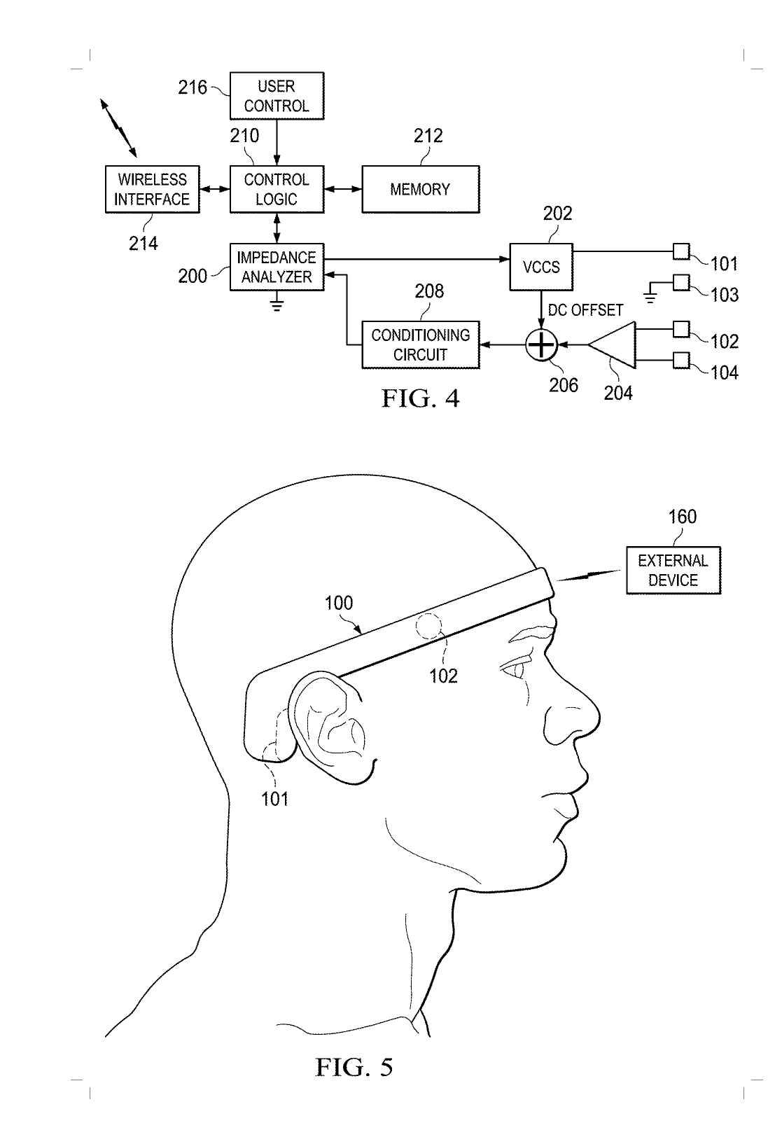

FIG. 4 shows an electrical block diagram of one embodiment of the CSF detection device 100. The electronics includes an impedance analyzer 200, a voltage controlled current source (VCCS) 202, an amplifier 204, a voltage summer 206, a conditioning circuit 208, control logic 210, memory 212, a wireless interface 214, and a user control (e.g., button). The control logic 210 may comprise an embedded controller that executes firmware stored in memory 212. The memory 212 may be separate from the control logic 210 or may be part of the control logic (e.g., memory internal to a controller). The control logic 210 may assert a signal to the impedance analyzer 200 to initiate an impedance measurement.

The impedance analyzer 200 may generate a signal at a known amplitude and frequency. The signal may be a fixed voltage that is provided to the VCCS 202 for generation of a current to electrode 101. The VCCS 202 may condition the current to remove any DC offset for safety reasons. The current passes through electrode 101, through the patient's cranium and returns via electrode 103, which may be grounded. As can be seen in FIGS. 1-3, electrodes 101 and 103, which represent the signal generation electrode pair, are interleaved with electrode pair 102 and 104, which represents the sense electrodes. That is, one temple tip electrode (e.g., 101) and one temple portion electrode (e.g., 103) are used as the signal generation electrode pair, and the temple portion electrode 102 is positioned circumferentially around the headband between the opposite temple tip electrode 104 and the temple portion electrode 102 closest to the signal generation electrode 101.

The amplifier 204 is coupled to the sensing electrodes 102 and 104 and amplifies the potential difference detected by those electrodes. In some embodiments, the amplifier 204 may comprise an instrumentation amplifier. The amplified signal is added by the voltage summer 206 with a voltage indicative of the DC offset removed from the generated signal by the VCCS 202. Thus, the DC offset is added to the received and amplified signal to replace the DC offset that was blocked initially by the VCCS 202.

Additional conditioning circuit 208 also may be included. Such conditioning circuit may include an inverting amplifier with a gain of, for example, 1, a second voltage controlled current source, a filter, etc. The resulting signal is provided back to the impedance analyzer 200. The impedance analyzer may cause a fixed amplitude current to be injected into the patient and measure the resulting voltage, or cause a fixed amplitude voltage to be applied to the patient and measure the resulting current. Either way, the ratio of voltage to current is impedance.

The calculation of impedance may be performed by the impedance analyzer 200 or the values of voltage and current may be provided to the control logic 210 for computation of impedance by the control logic. The calculated impedance may be stored in memory 212, and thus a log of impedance values may be stored in memory representing an impedance history over time for the patient. The history may be a day's worth of impedance data, or a week, a month, etc. The device may have a wireless interface (WiFi, Bluetooth, etc.) 214 to transmit the most recently computed impedance value or the impedance history log from memory 212 to an external device.

In some embodiments, the signal injected into the patient is an AC signal at a particular frequency. In such embodiments, the frequency may be swept from a first frequency to a second frequency. In some embodiments, the first and second frequencies may span a range of 1 KHz to 110 KHz. In one example, the first and second frequencies include 1 KHz and 110 MHz, respectively, and the frequency range is swept in 1, 3, or 10 KHz steps. At each such frequency (1 KHz, 4 KHz, 8 KHz, etc.), an impedance measurement is made. The resulting set of impedance measurements may be averaged to produce a final impedance measurement. The final impedance measurement may be a weighted average of the individual impedance measurements at each frequency, or may be an unweighted average.

FIG. 5 illustrates that the CSF detection device 100 can be in wireless communication with an external device 160. The external device 160 may comprise a smart phone, a tablet device, a computer (notebook, desktop, etc.), a bedside monitor or any other type of electronic device. The external device may include a display and permit a user to view the impedance data transmitted by the CSF detection device 100.

In some embodiments, the CSF detection device 100 determines that an excessive accumulation of CSF has occurred and sends a signal to the external device 160 accordingly, while in other embodiments the external device 160 makes that determination based on the transmission of raw data from the CSF detection device 100 (voltage and current data, impedance data, etc.).

The CSF detection device is calibrated to each patient initially while the patient's CSF drainage device (e.g., VP shunt) is working satisfactorily and has not failed. The calibration process may be initiated by a user activating the user control 216 on the CSF detection device. During the calibration mode, the CSF detection device 100 takes one or more impedance readings. If multiple readings are taken, such readings may be averaged together to produce a baseline impedance value. That value is stored in memory 212 and used for comparison purposes to determine if an excess of amount of CSF is accumulating in the patient's ventricles.

As CSF accumulates, cranial impedance will decrease. Once the measured impedance drops below a threshold, it is determined that excessive CSF has accumulated in the patient. The threshold may be a percentage of the baseline impedance measurement (e.g., 80% of the baseline). The comparison of a new impedance measurement to the threshold may be performed by the impedance analyzer 200, the control logic 210, or the external device 160. An alert may then be generated such as an audible and/or visual alert. The alert may include a text message or email sent to the patient, the patient's physician, or other person designated by the patient.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis. Further, in the following discussion and in the claims, the term “fluid” is defined to include blood and other types of body fluids or gases that may bleed or leak from a vessel or organ. All references to an impedance measurement being made encompasses any of the variations described herein as performed by the combination of the impedance assessment unit and an external apparatus.

What is claimed is:

1. A cerebrospinal fluid (CSF) detection device, comprising:
a headband including at least four electrodes integrated into the headband, wherein the electrodes are configured to make electrical contact with a person's head when the headband is placed on the person's head;
an impedance circuit integrated into the headband and coupled to the at least four electrodes, wherein the impedance circuit includes memory and an impedance analyzer coupled to the at least four electrodes and is configured to measure an impedance of the person's head;
wherein a first pair of the at least four electrodes is used by the impedance circuit to inject an electrical signal into the person's head and a second pair of the electrodes are used by the impedance analyzer to sense a signal, wherein the first pair of electrodes is interleaved within the headband with respect to the second pair of electrodes.

2. The CSF detection device of claim 1, further comprising a wireless interface.

3. The CSF detection device of claim 2, wherein the wireless interface is configured to transmit impedance measurements.

4. The CSF detection device of claim 2, wherein the wireless interface is configured to transmit impedance voltage and current data.

5. The CSF detection device of claim 2, wherein the memory is configured to store multiple impedance measurements and wherein the wireless interface is configured to transmit the stored impedance measurements to an external device.

6. The CSF detection device of claim 5, wherein the memory is configured to store impedance measurements taken over a time period of at least a month.

7. The CSF detection device of claim 1, wherein the headband includes a curved portion comprising a forehead segment, a pair of temple portions, and a pair of temple tips, wherein each temple tip includes one of the electrodes, and wherein a longitudinal axis of each temple tip is at an angle with respect to a longitudinal axis of each temple portion.

8. The CSF detection device of claim 6, wherein the angle is between about 80 degrees and about 120 degrees.

9. The CSF detection device of claim 8, wherein each temple portion includes one of the electrodes.

10. The CSF detection device of claim 1, wherein the headband is not one continuous loop and, instead, is configured to fit about a partial circumference of the person's head.

11. A cerebrospinal fluid (CSF) detection device, comprising:
a headband including at least four electrodes integrated into the headband, wherein the electrodes are configured to make electrical contact with a person's head when the headband is placed on the person's head;
an impedance circuit integrated into the headband and coupled to the at least four electrodes, wherein the impedance circuit includes memory and an impedance analyzer coupled to the at least four electrodes and is configured to measure an impedance of the person's head;
wherein a first pair of the at least four electrodes is used by the impedance circuit to inject an electrical signal into the person's head and a second pair of the electrodes are used by the impedance analyzer to sense a signal, wherein the first pair of electrodes is interleaved within the headband with respect to the second pair of electrodes;
wherein the impedance circuit is configured to detect an accumulation of cerebrospinal fluid based on a comparison of a measured impedance of the person's head to a baseline impedance value.

12. The CSF detection device of claim 11, wherein the headband includes a curved portion comprising a forehead segment, a pair of temple portions, and a pair of temple tips, wherein each temple tip includes one of the electrodes, and wherein a longitudinal axis of each temple tip is at an angle with respect to a longitudinal axis of each temple portion.

13. The CSF detection device of claim 12, wherein the angle is between about 80 degrees and about 120 degrees.

14. The CSF detection device of claim 13, wherein each temple portion includes one of the electrodes.

15. The CSF detection device of claim 11, wherein the headband is not one continuous loop and, instead, is configured to fit about a partial circumference of the person's head.
